(19) United States
(12) Patent Application Publication
(10) Pub. No.: US 2025/0229018 A1
(43) Pub. Date: Jul. 17, 2025

(54) DRUG INFUSION DEVICE WITH INTEGRATED POWER SUPPLY AND ARTIFICIAL PANCREAS

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/703,719

(22) PCT Filed: Dec. 28, 2021
(86) PCT No.: PCT/CN2021/141895

§ 371 Date: Apr. 23, 2024

(51) Int. Cl. A61M 5/142; A61M 5/145; A61M 5/315; H01M 10/42; H01M 50/103; H01M 50/121; H01M 50/124; H01M 50/176; H01M 50/186; H01M 50/191; H01M 50/198; H01M 50/202; H01M 50/247; H01M 50/271; H01M 50/284

(52) U.S. Cl. CPC A61M 5/14248; A61M 5/1452; A61M 5/31528; H01M 10/425; H01M 50/103; H01M 50/121; H01M 50/1243; H01M 50/176; H01M 50/186; H01M 50/191; H01M 50/198; H01M 50/202; H01M 50/247; H01M 50/271; H01M 50/284; A61M 2205/8206; H01M 2220/30

(57) ABSTRACT

A drug infusion device with integrated power supply includes a drug reservoir, used for accommodating the drug to be infused, provided with a piston and a screw; a driving wheel, connected with the screw, driving the screw to push the piston forward by rotation; a power supply, used to supply power to the infusion device, including a power supply shell, a power supply cell, electrolyte and a cover plate; and a case, including an upper case and a lower case, for accommodating the drug reservoir, the drive wheel and the power supply. The power supply shell is integrated with the lower and/or the cover plate is integrated with the upper case.

### TECHNICAL FIELD

The present invention mainly relates to the field of medical instruments, in particular to a drug infusion device with integrated power supply and the artificial pancreas thereof.

### BACKGROUND

In a healthy person, the pancreas can automatically monitor the amount of glucose in the blood and automatically secrete the required dosage of insulin/glucagon. However, for diabetic patients, the function of their pancreas has been severely compromised, and the pancreas cannot secrete the required dosage of insulin. Therefore, diabetes mellitus is defined as a metabolic disease caused by abnormal pancreatic function, and it is also classified as one of the top three chronic conditions by the WHO. The present medical advancement has not been able to find a cure for diabetes mellitus. Yet, the best the technology could do is control the onset symptoms and complications by stabilizing the blood glucose level for diabetes patients.

Diabetic patients on an insulin pump need to check their blood glucose before infusing insulin into their bodies. At present, most detection methods can continuously detect blood glucose and send the blood glucose data to the remote device in real-time for the user to view. This detection method is called Continuous Glucose Monitoring (CGM), which requires the detection device to be attached to the surface of the patients' skin, and the sensor carried by the device to be inserted into the interstitial fluid for testing. According to the blood glucose (BG) level, the infusion system mimics an artificial pancreas to fill the gaps of the required insulin amount via the closed-loop pathway or the semi-closed-loop pathway.

However, the power supply of the current infusion device is a button battery. Because the limited storage capacity of a single button battery and the large volume ratio of the battery shell, usually, multiple button batteries are required to meet the working requirements of the infusion device. Therefore, a certain space for multiple button batteries must be reserved in the infusion device, leads to the low volume utilization rate of the infusion device, which increases the design difficulty for miniaturization of the infusion device.

Therefore, in the prior art, there is an urgent need for a drug infusion device with a power supply with a smaller volume and a larger capacity.

### BRIEF SUMMARY OF THE INVENTION

The invention discloses a drug infusion device with integrated power supply, the power supply shell and the lower case are integrated and/or the cover plate and the upper case are integrated. The shape and size of the power supply are no longer restricted by the shape and size of the button battery shell, and there is no need for a separate shell. The intergrated power supply occupies a small volume, and more active materials can be accommodated to increase the battery capacity, which can further reduce the volume of the infusion device while meeting the long-term working requirements of the infusion device, and improve the user experience.

The invention discloses a drug infusion device with integrated power supply that includes a drug reservoir, used for accommodating the drug to be infused, provided with a piston and a screw; a driving wheel, connected with the screw, driving the screw to push the pistion forward by rotation; a power supply, used to supply power to the infusion device, including a power supply shell, a battery cell, electrolyte and a cover plate; a case, including an upper case and a lower case, for accommodating the drug reservoir, the drive wheel, and the power supply, the power supply shell is integrated with the lower case and/or the cover plate is integrated with the upper case.

According to one aspect of the present invention, an electrolyte isolation layer is arranged on the inside of the power supply shell and the cover plate.

According to one aspect of the present invention, the electrolyte isolation layer is a coated TPE or PET layer.

According to one aspect of the present invention, the thickness of the electrolyte isolation layer is 300 μm-500 μm.

According to one aspect of the present invention, the electrolyte isolation layer is a separated TPE or PET layer.

According to one aspect of the present invention, the junction between the power supply shell and the cover plate is coated with an insulating sealing material.

According to one aspect of the present invention, the insulating sealing material is hot melt glue or silica gel.

According to one aspect of the present invention, the battery cell includes a positive electrode sheet, a negative electrode sheet, a separator, a positive electrode tab and a negative electrode tab, the positive electrode tab is fixedly connected to the positive electrode sheet, the negative electrode tab is fixedly connected to the negative electrode sheet.

According to one aspect of the present invention, the infusion device further comprises a three dimensional circuit, and the positive electrode sheet tab and the negative electrode sheet tab are electrically connected to the three dimensional circuit, respectively, to realize power supply to the infusion device.

According to one aspect of the present invention, when the cover plate is covering on the power supply shell, a part of the positive electrode tab and the negative electrode tab are reserved out of the power supply shell to be electrically connected to the three dimensional circuit, respectively.

According to one aspect of the present invention, two small holes are provided on the power supply shell, and the positive electrode tab and the negative electrode tab are electrically connected to the three dimensional circuit, respectively, through the small holes.

According to one aspect of the present invention, the infusion device further comprises a frame at least for carrying the driving wheels.

According to one aspect of the present invention, the three dimensional circuit is coated on the frame.

According to one aspect of the present invention, the three dimensional circuit is embedded in the frame.

According to one aspect of the present invention, the three dimensional circuit is coated on the upper case and/or the lower case.

According to one aspect of the present invention, the three dimensional circuit is embedded in the upper case and/or the lower case.

According to one aspect of the present invention, the infusion device comprises an infusion mechanism module and a control mechanism module, the drug reservoir, the drive wheel, and the power supply are provided on the infusion mechanism module.

According to one aspect of the present invention, the infusion mechanism module and the control mechanism module are designed separately, and the control mechanism module can be reused.

According to one aspect of the present invention, the infusion mechanism module and the control mechanism module are disposed of in one housing, discarded together after a single-use.

The invention discloses an artificial pancreas, comprises a drug infusion device with integrated power supply, and a detection mechanism module, configured to detect blood glucose continuously, connected or integrated with the control mechanism module and the infusion mechanism module of the infusion device.

According to one aspect of the present invention, any two of the control mechanism module, infusion mechanism module and the detection mechanism module are connected or integrated with each other configured to form a single part whose attached position on the skin is different from the third module.

According to one aspect of the present invention, the control mechanism module, infusion mechanism module and the detection mechanism module are connected or integrated with each other configured to form a single part, which is attached on only one position on the skin.

Compared with the prior art, the technical solution of the present invention has the following advantages:

In the drug infusion device with integrated power supply disclosed by the present invention, the power supply shell and the lower case are integrated and/or the cover plate and the upper case are integrated. The shape and size of the power supply are no longer restricted by the shape and size of the button battery shell, and there is no need for a separate shell. The intergrated power supply occupies a small volume, and more active materials can be accommodated to increase the battery capacity, which can further reduce the volume of the infusion device while meeting the long-term working requirements of the infusion device, and improve the user experience.

Furthermore, the electrolyte isolation layer is a TPE or PET layer, which can effectively prevent the power supply shell and cover plate from being corroded by the electrolyte.

Furthermore, the power supply shell coated with hot melt glue, on the one hand, it can prevent the electrolyte from leakaging; On the other hand, it is helpful to the self-thermal runaway management of power supply.

Furthermore, further comprises a three dimensional circuit, according to the internal arrangement characteristics of the infusion device, the shape and position of the three dimensional circuit can be flexibly designed, which can make the full use of the internal space of the infusion mechanism module, making the arrangement more compact and reducing the volume of the infusion device.

Furthermore, the three dimensional circuit is embedded in the upper case and/or the lower case or the frame, which can further reduce the volume of the infusion device.

Furthermore, after a single use, the power supply is discarded with the infusion mechanism module, and the power supply is also replaced when the infusion mechanism module or infusion device is replaced, which can make the power supply always maintain a high-performance working state.

### BRIEF DESCRIPTION OF THE DRAWINGS

FIG. 1a and FIG. 1b are schematic top views of the drug infusion device according to two embodiments of the present invention.

FIG. 2a is a schematic view of the drug infusion device according to an embodiment of the present invention.

FIG. 2b a cross-sectional view of the power supply in the Y-Y′ direction according to the embodiment of the present invention.

FIG. 2c is schematic view of the drug infusion device from another perspective according to the embodiment of the present invention.

FIG. 3a is a schematic view of the drug infusion device according to another embodiment of the present invention.

FIG. 3b a cross-sectional view of the power supply in the Y-Y′ direction according to the embodiment of the present invention.

FIG. 3c is schematic view of the drug infusion device from another perspective according to the embodiment of the present invention.

FIG. 4 is a schematic view of the module relationship of the artificial pancreas according to one embodiment of the present invention.

### DETAILED DESCRIPTION

As mentioned above, in the prior art, the button battery shell in infusion device occupies a large volume ratio and the battery capacity is small, which leads to low volume utilization rate of the infusion device, and increases the design difficulty of miniaturization of the infusion device.

In order to solve this problem, the present invention provides a drug infusion device, the power supply shell and the lower case are integrated and/or the cover plate and the upper case are integrated. The shape and size of the power supply are no longer restricted by the shape and size of the button battery shell, and there is no need for a separate shell. The intergrated power supply occupies a small volume, and more active materials can be accommodated to increase the battery capacity and the volume utilization rate of the infusion device, which can further reduce the volume of the infusion device while meeting the long-term working requirements of the infusion device, and improve the user experience.

Various exemplary embodiments of the present invention will now be described in detail regarding the figures. The relative arrangement of the components and the steps, numerical expressions and numerical values outlined in the embodiments are not construed as limiting the scope of the invention.

In addition, it should be understood that, for ease of description, the dimensions of the various components shown in the figures are not necessarily drawn in the actual scale relationship; for example, the thickness, width, length or distance of certain units may be exaggerated relative to other mechanism modules.

The following description of the exemplary embodiments is merely illustrative and does not limit the invention its application or use. The techniques, methods, and devices are known to those of ordinary skill in the art and may not be discussed in detail. However, such techniques, methods, and devices should be considered as part of the specification.

It should be noted that similar reference numerals and letters indicate similar items in the following figures. Therefore, once an item is defined or illustrated in a drawing, it will not be discussed further in the following description of the drawings.

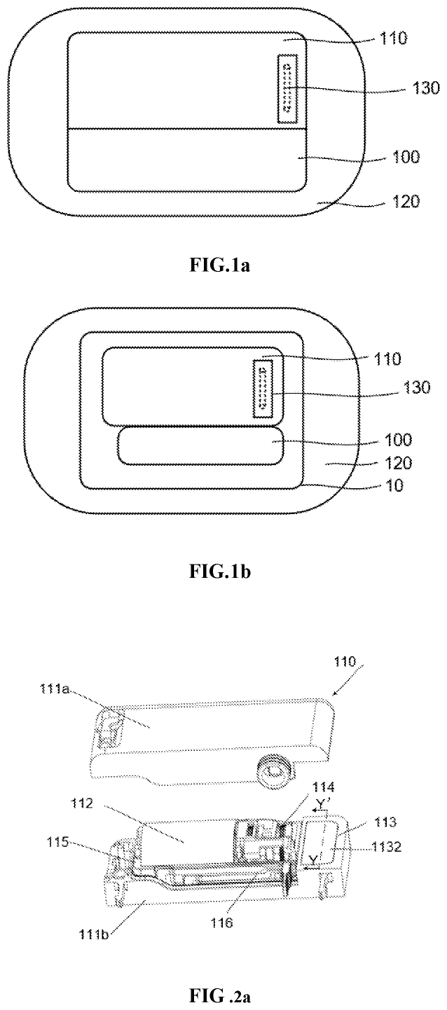

FIG. 1a and FIG. 1b are schematic top views of the drug infusion device according to two embodiments of the present invention.

In the embodiment of the present invention, the highly integrated drug infusion device comprises a control mechanism module 100, an infusion mechanism module 110 and an adhesive patch 120, which will be described separately below. In other embodiments of the present invention, the patch-type drug infusion device may include more parts, which are not specifically limited here.

The patch-type drug infusion device refers to a tubing-free infusion device that is entirely pasted on the user's skin surface by the one piece of adhesive patch 120. And the infusion device is provided with an infusion needle unit 130, integrated on the infusion device, instead of a long tube; therefore, the drug can be directly infused from the drug reservoir to the subcutaneous tissue through the infusion needle unit 130.

The highly integrated drug infusion device of the embodiment of the present invention includes a control mechanism module 100, which receives signals or information from a remote device or a body fluid parameter detection device (such as CGM), and controls the infusion device to infuse drug(s) accordingly. Inside the housing 101 of the control mechanism module 100 are disposed of program modules, circuit board(s) and related electronic units for receiving signals or issuing control instructions, as well as other mechanical units or components necessary for realizing the infusion function, which is not limited herein. In another embodiment of the present invention, a power supply 113 can also be provided in the control mechanism module. Preferably, in the embodiment of the present invention, the power supply 113 is provided in the infusion mechanism module 110, which will be described below.

The highly integrated drug infusion device further includes an infusion mechanism module 110 with a case. A mechanical module, an electric control module, and other auxiliary modules for completing the drug infusion process are provided inside the case, which will be described in detail below. The case of the infusion mechanism module 110 may include multiple parts. As in the embodiment of the present invention, the case of the infusion system includes an upper case 111a and a lower case 111b.

In the embodiment of the present invention, the infusion mechanism module 110 and the control mechanism module 100 are designed separately and connected by a waterproof plug or directly engaged and electrically connected into a whole. The infusion mechanism module 110 can be reused, and the control mechanism module 100 is discarded after a single use, as shown in FIG. 1a. In another embodiment of the present invention, the infusion mechanism module 110 and the control mechanism module 100 are connected by a wire and disposed of inside the same housing 10. Attached to a certain position of the user's skin by the adhesive patch 120, both units will be discarded together after a single use, as shown in FIG. 1b.

The highly integrated drug infusion device further includes a needle unit 130, used for infusing the drug to the subcutaneous tissue.

The adhesive patch 120 is also provided on the bottom of the lower case 111b to attach the infusion device to the user's skin surface.

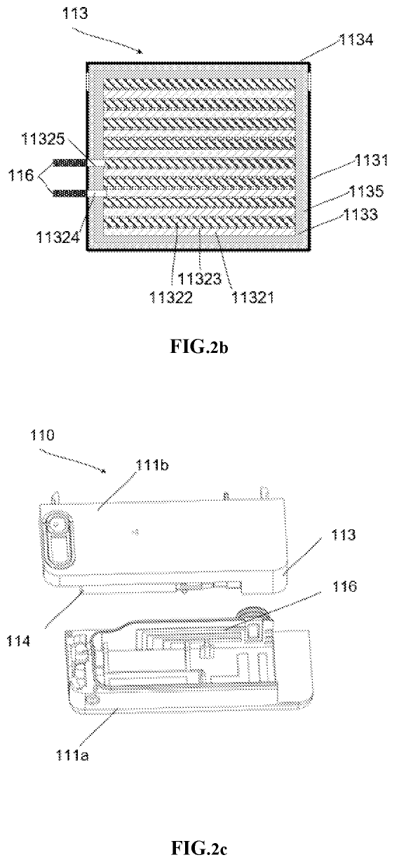

FIG. 2a is a schematic view of the drug infusion device according to an embodiment of the present invention. FIG. 2b a cross-sectional view of the power supply in the Y-Y′ direction according to the embodiment of the present invention. FIG. 2c is schematic view of the drug infusion device from another perspective according to the embodiment of the present invention.

In the embodiment of the present invention, the infusion mechanism module 110 includes mechanical units and electronic control units used to realize the infusion function, such as a drug reservoir 112, a power supply 113, a driving wheel 114, a frame 115, a three dimensional circuit 136, a driving unit (not shown), etc. The movement of the driving unit drives the driving wheel 114 to rotate, thus making the screw (not shown) push the piston (not shown) in the drug reservoir 112 forward, realizing the drug infusion.

In the embodiment of the present invention, the power supply 113 includes a power supply shell 1131, a battery cell 1132, electrolyte 1133 and a cover plate 1134. Put the battery cell 1132 into the power supply shell 1131 and inject the electrolyte 1133 from the opening of the shell 1131, then cover the cover plate 1134, and coat the insulating sealing material at the junction of the cover plate 1134 and the shell 1131. In the embodiment of the present invention, the insulating sealing material is hot melt glue or silica gel. Preferably, the insulating sealing material is hot melt glue, on the one hand, it can prevent the electrolyte from leakaging; On the other hand, it is helpful to the self-thermal runaway management of power supply. In another embodiment of the present invention, the sealing can also be performed in other ways, such as adding a gasket at the cover plate 1134. The specific sealing method is not specifically limited here, as long as the power supply 113 can be sealed to prevent electrolyte from leakaging.

In the embodiment of the present invention, the power supply shell 1131 and lower case 111b of the infusion device are integrated, and lower case 111b is a conventional plastic part, such as PE (polyethylene), PP (polypropylene), PC (polycarbonate)), easy to be corroded by electrolyte, so its inner surface is coated with electrolyte isolating layer 1135, such as spraying PET (polyethylene terephthalate) or TPE (butyl rubber) material, PET and TPE are corrosion-resistant material of electrolyte, which can effectively isolate damage to the power supply shell 1131 and circuit components by the electrolyte. The thickness of the electrolyte isolation layer is 300 μm-500 μm. If the thickness is too thin, the PET film will be infiltrated and softened by the electrolyte. When the amount of electrolyte is small, although the PET film will not dissolve and penetrate, the isolation effect will still exist, but for too long, it may cause the device to deteriorate. While excessive thickness will increase the weight and volume of the power supply shell 1131, which is not conducive to the miniaturization of the infusion device.

In another embodiment of the present invention, the power supply shell 1131 can also be layered, that is, the inner and outer layers are made of different materials, and the outer layer is conventional plastic, such as the aforementioned PE, PP, PC, etc., and the inner layer is TPE (butyl Rubber) or PET (polyethylene terephthalate) layer. TPE is a thermoplastic elastomer material with strong processability and can prevent electrolyte corrosion; PET itself can be used as a container for electrolyte and is resistant to electrolyte corrosion. Both TPE and PET can effectively isolate the electrolyte from damaging the power supply shell 1131 and circuit components.

Similarly, the electrolyte isolation layer 1135 is also provided on the inside of the cover plate 1134. Preferably, the electrolyte isolation layer 1135 on the inside of the cover plate 1134 is arranged in the same manner as the power supply shell 1131.

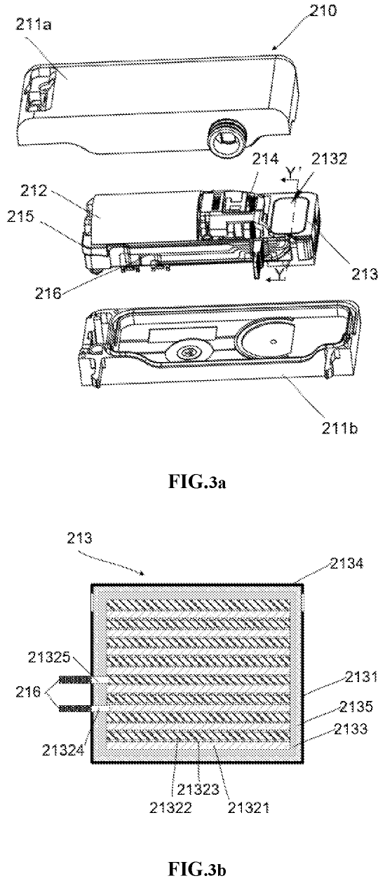

In another embodiment of the present invention, the cover plate 1134 and the upper case 111a of the infusion device (as shown in FIG. 3a) are integrated, and the upper case 111a of the infusion device is a conventional plastic part, such as PE (Polyethylene), PP (polypropylene), PC (polycarbonate), which are easily corroded by electrolyte, so the inner surface is coated with electrolyte isolation layer 1135, such as spraying PET or TPE material, or layered PET or TPE layer.

It should be noted that in the embodiment of the present invention, “upper case” and “lower case” are only relative concepts, that is, the power supply shell 1131 can also be an integrated with the upper case 111a, and the cover plate 1134 can also be an integrated with the lower case 111b.

In the embodiment of the present invention, the power supply shell 1131 and lower case 111b, the cover plate 1134 and the upper case 111a may be an integrated at the same time, or may be integrated, respectively. When only one are integrated, for example, when the power supply shell 1131 and lower case 111b are integrated, the cover plate 1134 can be independent of the upper case 111a; when the cover plate 1134 and the upper case 111a are integrated, the power supply shell 1131 can be independent of the lower case 111b. When the power supply shell 1131 and lower case 111b, the cover 1134 and the upper case 111a are integrated at the same time, for the junction which is located inside the infusion device, before the cover plate 1134 is covered on the power supply shell 1131, the junction can be coated with insulating sealing material, such as hot melt glue, and after the cover plate 1134 is covered on the power supply shell 1131, the hot melt adhesive can be bonded to the cover plate 1134 and the power supply shell 1131 by external heating, such as infrared heating or ultraviolet heating. And for the junction located outside the infusion device, the insulating sealing material can be coated after the cover plate 1134 is covered on the power supply shell 1131.

In the embodiment of the present invention, the electrolyte 1133 is one of ethylene carbonate, propylene carbonate, diethyl carbonate, dimethyl carbonate, ethyl methyl carbonate, lithium hexafluorophosphate, phosphorus pentafluoride, or hydrofluoric acid.

In the embodiment of the present invention, the material of the separator 11323 is PE (polyethylene) or PP (polypropylene), which may be a single layer of PE or PP, or a three-layer of PE or PP.

In the embodiment of the present invention, the battery cell 1132 is a wound cell or a laminated cell. The specific type of the cell can be selected according to the shape of the power supply shell 1131. When the power supply shell 1131 is cylindrical, the battery cell is a wound cell. When the power supply shell 1131 is square, the battery cell is a square laminated cell. When the power supply shell 1131 is of other special shapes, the corresponding battery cell can also be a special-shaped battery cell. There is no specific limitation, as long as the internal space of the power supply shell 1131 can be fully utilized, the electrode active material is filled to the greatest extent, and increase the battery capacity, so that the capacity of the power supply 113 is increased compared with the button battery, and increase the life time of the infusion device.

The battery cell 1132 includes a positive electrode sheet 11321, a negative electrode sheet 11322, a separator 11323, a positive electrode tab 11324, and a negative electrode tab 11325. One end of the positive electrode tab 11324 is fixedly connected to the positive electrode sheet 11321. Preferably, it is connected by soldering or solder paste, and the other end is electrically connected to an external circuit through a small hole provided in the shell 11321. The specific electrical connection method will be detailed below. The opening and size of the small hole are adapted to the cross-sectional shape and size of the positive electrode tab 11324, and at the same time, an insulating sealing material is coated at the power supply shell where the opening located, to ensure complete sealing and no electrolyte penetration. Preferably, the sealing material is hot melt glue, and the hot melt glue can be helpful to the self-thermal runaway management of the power supply 113 while ensuring complete sealing.

Similarly, one end of the negative electrode tab 11325 is fixedly connected to the negative electrode sheet 11322. Preferably, it is connected by soldering or solder paste, and the other end is electrically connected to an external circuit through a small hole provided on the shell 1131, and at the same time, hot melt glue is coated at the power supply shell where the small hole located.

In another embodiment of the present invention, the positive electrode tab 11324 and the negative electrode tab 11325 may not be electrically connected to the outside through the small hole, but when covering the cover plate 1134, a part of the electrode tab is reserved outside the power supply shell 1131, used for electrical connection with an external circuit, and a sealing material is coated at the junction of the cover plate 1134 and the shell 1131. Preferably, the sealing material is hot melt glue.

In the embodiment of the present invention, the material of the positive electrode tab 11324 is aluminum, and the material of the negative electrode tab 11325 is nickel or copper plated with nickel.

In the embodiment of the present invention, the positive electrode material on the positive electrode sheet 11321 may be manganese dioxide, and the corresponding negative electrode material of 11322 is metal lithium and other lithium-based materials. In other embodiments of the present invention, the positive electrode material may be lithium manganate, lithium cobaltate, lithium iron phosphate and other lithium-containing compounds, the corresponding negative electrode material is graphite.

The infusion mechanism module 110 in the embodiment of the present invention is also provided with a three dimensional circuit 116, supplying power to specific units by being connected to the positive electrode tab 11324 and the negative electrode tab 11325 respectively. According to the internal arrangement characteristics of the infusion device, the shape and position of the three dimensional circuit can be flexibly designed, which can make the full use of the internal space of the infusion mechanism module, making the arrangement more compact.

In the embodiment of the present invention, the three dimensional circuit 116 is disposed on the frame 115, as shown in FIG. 2a. In an embodiment of the present invention, the three dimensional circuit 116 is a three dimensional printed circuit coated on the frame 115. In another embodiment, the three dimensional circuit 116 is embedded in the frame 115. For example, the frame 115 is provided with a groove for accommodating the three dimensional circuit 116, and the three dimensional circuit 116 is embedded in the frame 115 through the groove, or the three dimensional circuit 116 is integrated into the frame 115 by injection molding, which can further reduce the volume of the infusion device.

In another embodiment of the present invention, the three dimensional circuit 116 is disposed on the upper case 111a, as shown in FIG. 2c. In an embodiment of the present invention, the three dimensional circuit 116 is a three dimensional printed circuit coated inside the upper case 111a. In another embodiment of the present invention, the three dimensional circuit is embedded in the upper case 111a. For example, the upper case 111a is provided with a groove for accommodating the three dimensional circuit 116, and the three dimensional circuit 116 is embedded in the upper case through the groove. In 111a, or the three dimensional circuit 116 and the upper case 111a are integrated by injection molding. When the three dimensional circuit 116 is coated or embedded in the upper case 111a, the reservoir 112 is directly fixed in the case, the specific fixing method is not limited here, without the frame 115 to carry, which can greatly reduce the weight and volume of the infusion device by reducing part of the frame 115. When the three dimensional circuit 116 is embedded in the upper case 111a, the weight and volume of the infusion device can be further reduced.

In another embodiment of the present invention, the three dimensional circuit 116 may also be arranged on the lower case 111b, or arranged on the upper case 111a and the lower case 111b at the same time, and the detail arrangement and beneficial effects are the same as that of the three dimensional circuit 116 being arranged on the upper case 111a, which won't be repeated it here. In particular, when the three dimensional circuit 116 is disposed on the upper case 111a and the lower case 111b at the same time, a part of the three dimensional circuit 116 is disposed on the upper case 111a, and another part of the three dimensional circuit 116 is disposed on the lower case 111b.

FIG. 3a is a schematic view of the drug infusion device according to an embodiment of the present invention. FIG. 3b a cross-sectional view of the power supply in the Y-Y′ direction according to the embodiment of the present invention. FIG. 3c is schematic view of the drug infusion device from another perspective according to the embodiment of the present invention.

In the embodiment of the present invention, the power supply 213 includes a power supply shell 2131, a battery cell 2132, electrolyte 2133 and a cover plate 2134. Put the battery cell 2132 into the power supply shell 2131 and inject the electrolyte 2133 from the opening of the shell 2131, then cover the cover plate 2134, and coat the insulating sealing material at the junction of the cover plate 2134 and the shell 2131. In the embodiment of the present invention, the insulating sealing material is hot melt glue or silica gel. Preferably, the insulating sealing material is hot melt glue, on the one hand, it can prevent the electrolyte from leakaging; On the other hand, it is helpful to the self-thermal runaway management of power supply. In another embodiment of the present invention, the sealing can also be performed in other ways, such as adding a gasket at the cover plate 2134. The specific sealing method is not specifically limited here, as long as the power supply 213 can be sealed to prevent electrolyte from leakaging.

In the embodiment of the present invention, the power supply shell 2131 and the frame 215 of the infusion device are integrated, and the frame 215 is a conventional plastic part, such as PE (polyethylene), PP (polypropylene), PC (polycarbonate)), easy to be corroded by electrolyte, so its inner surface is coated with electrolyte isolating layer 2135, such as spraying PET (polyethylene terephthalate) or TPE (butyl rubber) material, PET and TPE are corrosion-resistant material of electrolyte, which can effectively isolate damage to the power supply shell 2131 and circuit components by the electrolyte. The thickness of the electrolyte isolation layer is 300 μm-500 μm. If the thickness is too thin, the PET film will be infiltrated and softened by the electrolyte. When the amount of electrolyte is small, although the PET film will not dissolve and penetrate, the isolation effect will still exist, but for too long, it may cause the device to deteriorate. While excessive thickness will increase the weight and volume of the power supply shell 2131, which is not conducive to the miniaturization of the infusion device.

In another embodiment of the present invention, the power supply shell 2131 can also be layered, that is, the inner and outer layers are made of different materials, and the outer layer is conventional plastic, such as the aforementioned PE, PP, PC, etc., and the inner layer is TPE (butyl Rubber) or PET (polyethylene terephthalate) layer. TPE is a thermoplastic elastomer material with strong processability and can prevent electrolyte corrosion; PET itself can be used as a container for electrolyte and is resistant to electrolyte corrosion. Both TPE and PET can effectively isolate the electrolyte from damaging the power supply shell 2131 and circuit components.

Similarly, the electrolyte isolation layer 2135 is also provided on the inside of the cover plate 2134. Preferably, the electrolyte isolation layer 2135 on the inside of the cover plate 2134 is arranged in the same manner as the power supply shell 2131.

In another embodiment of the present invention, the cover plate 2134 and the upper case 211a or the lower case 211b of the infusion device are integrated, and the upper case 211a or the lower case 211b of the infusion device is a conventional plastic part, such as PE (Polyethylene), PP (polypropylene), PC (polycarbonate), which are easily corroded by electrolyte, so the inner surface is coated with electrolyte isolation layer 2135, such as spraying PET or TPE material, or layered PET or TPE layer.

In the embodiment of the present invention, the power supply shell 2131 and the frame 215, the cover plate 2134 and the upper case 211a or the lower case 211b may be an integrated at the same time, or may be integrated, respectively. When only one are integrated, for example, when the power supply shell 2131 and the frame 215 are integrated, the cover plate 2134 can be independent of the upper case 211a or the lower case 211b; when the cover plate 2134 and the upper case 211a or the lower case 211b are integrated, the power supply shell 2131 can be independent of the frame 215. When the power supply shell 2131 and the frame 215, the cover 2134 and the upper case 211a or the lower case 211b are integrated at the same time, for the junction which is located inside the infusion device, before the cover plate 2134 is covered on the power supply shell 2131, the junction can be coated with insulating sealing material, such as hot melt glue, and after the cover plate 2134 is covered on the power supply shell 2131, the hot melt adhesive can be bonded to the cover plate 2134 and the power supply shell 2131 by external heating, such as infrared heating or ultraviolet heating. And for the junction located outside the infusion device, the insulating sealing material can be coated after the cover plate 2134 is covered on the power supply shell 2131.

In the embodiment of the present invention, the electrolyte 2133 is one of ethylene carbonate, propylene carbonate, diethyl carbonate, dimethyl carbonate, ethyl methyl carbonate, lithium hexafluorophosphate, phosphorus pentafluoride, or hydrofluoric acid.

In the embodiment of the present invention, the material of the separator 21323 is PE (polyethylene) or PP (polypropylene), which may be a single layer of PE or PP, or a three-layer of PE or PP.

In the embodiment of the present invention, the battery cell 2132 is a wound cell or a laminated cell. The specific type of the cell can be selected according to the shape of the power supply shell 2131. When the power supply shell 2131 is cylindrical, the battery cell is a wound cell. When the power supply shell 2131 is square, the battery cell is a square laminated cell. When the power supply shell 2131 is of other special shapes, the corresponding battery cell can also be a special-shaped battery cell. There is no specific limitation, as long as the internal space of the power supply shell 2131 can be fully utilized, the electrode active material is filled to the greatest extent, and increase the battery capacity, so that the capacity of the power supply 213 is increased compared with the button battery, and increase the life time of the infusion device.

The battery cell 2132 includes a positive electrode sheet 21321, a negative electrode sheet 21322, a separator 21323, a positive electrode tab 21324, and a negative electrode tab 21325. One end of the positive electrode tab 21324 is fixedly connected to the positive electrode sheet 21321. Preferably, it is connected by soldering or solder paste, and the other end is electrically connected to an external circuit through a small hole provided in the shell 2131. The specific electrical connection method will be detailed below. The opening and size of the small hole are adapted to the cross-sectional shape and size of the positive electrode tab 21324, and at the same time, an insulating sealing material is coated at the power supply shell where the opening located, to ensure complete sealing and no electrolyte penetration. Preferably, the sealing material is hot melt glue, and the hot melt glue can be helpful to the self-thermal runaway management of the power supply 213 while ensuring complete sealing.

Similarly, one end of the negative electrode tab 21325 is fixedly connected to the negative electrode sheet 21322. Preferably, it is connected by soldering or solder paste, and the other end is electrically connected to an external circuit through a small hole provided on the shell 2131, and at the same time, hot melt glue is coated at the power supply shell where the small hole located.

In another embodiment of the present invention, the positive electrode tab 21324 and the negative electrode tab 21325 may not be electrically connected to the outside through the small hole, but when covering the cover plate 2134, a part of the electrode tab is reserved outside the power supply shell 2131, used for electrical connection with an external circuit, and a sealing material is coated at the junction of the cover plate 2134 and the shell 2131. Preferably, the sealing material is hot melt glue.

In the embodiment of the present invention, the material of the positive electrode tab 21324 is aluminum, and the material of the negative electrode tab 21325 is nickel or copper plated with nickel.

In the embodiment of the present invention, the positive electrode material on the positive electrode sheet 21321 may be manganese dioxide, and the corresponding negative electrode material of 21322 is metal lithium and other lithium-based materials. In other embodiments of the present invention, the positive electrode material may be lithium manganate, lithium cobaltate, lithium iron phosphate and other lithium-containing compounds, the corresponding negative electrode material is graphite.

The infusion mechanism module 110 in the embodiment of the present invention is also provided with a three dimensional circuit 216, supplying power to specific units by being connected to the positive electrode tab 21324 and the negative electrode tab 21325 respectively. According to the internal arrangement characteristics of the infusion device, the shape and position of the three dimensional circuit can be flexibly designed, which can make the full use of the internal space of the infusion mechanism module, making the arrangement more compact.

In the embodiment of the present invention, the three dimensional circuit 216 is disposed on the frame 215, as shown in FIG. 3a. In an embodiment of the present invention, the three dimensional circuit 216 is a three dimensional printed circuit coated on the frame 215. In another embodiment, the three dimensional circuit 216 is embedded in the frame 215. For example, the frame 215 is provided with a groove for accommodating the three dimensional circuit 216, and the three dimensional circuit 216 is embedded in the frame 215 through the groove, or the three dimensional circuit 216 is integrated into the frame 215 by injection molding, which can further reduce the volume of the infusion device.

In another embodiment of the present invention, the three dimensional circuit 216 is disposed on the upper case 211a, as shown in FIG. 3c. In an embodiment of the present invention, the three dimensional circuit 216 is a three dimensional printed circuit coated inside the upper case 211a. In another embodiment of the present invention, the three dimensional circuit is embedded in the upper case 211a. For example, the upper case 211a is provided with a groove for accommodating the three dimensional circuit 216, and the three dimensional circuit 216 is embedded in the upper case through the groove. In 211a, or the three dimensional circuit 216 and the upper case 211a are integrated by injection molding. When the three dimensional circuit 216 is coated or embedded in the upper case 211a, the reservoir 212 is directly fixed in the case, the specific fixing method is not limited here, without the frame 215 to carry, which can greatly reduce the weight and volume of the infusion device by reducing part of the frame 215. When the three dimensional circuit 216 is embedded in the upper case 211a, the weight and volume of the infusion device can be further reduced.

In another embodiment of the present invention, the three dimensional circuit 216 may also be arranged on the lower case 211b, or arranged on the upper case 211a and the lower case 211b at the same time, and the detail arrangement and beneficial effects are the same as that of the three dimensional circuit 216 being arranged on the upper case 211a, which won't be repeated it here. In particular, when the three dimensional circuit 216 is disposed on the upper case 211a and the lower case 211b at the same time, a part of the three dimensional circuit 216 is disposed on the upper case 211a, and another part of the three dimensional circuit 216 is disposed on the lower case 211b.

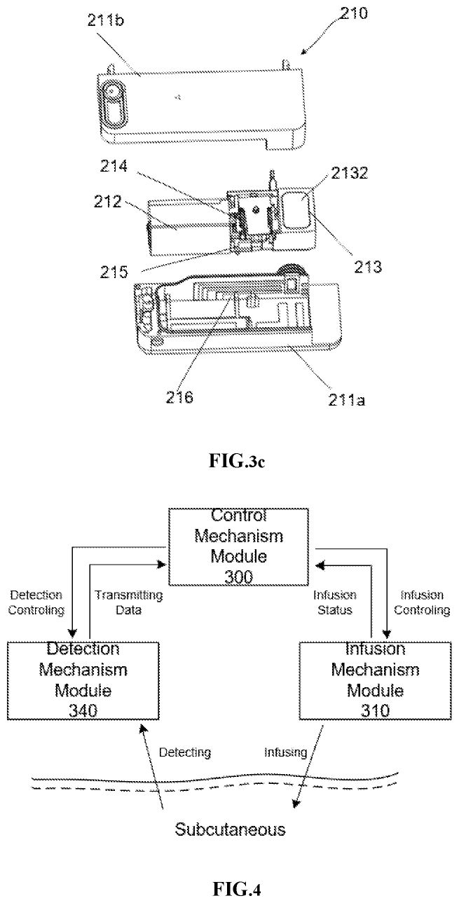

FIG. 4 is a schematic view of the module relationship of the artificial pancreas according to one embodiment of the present invention.

The artificial pancreas disclosed in the embodiment of the present invention comprises the above mentioned infusion device with integrated power supply; and a detection mechanism module 340, connected or integrated with the control mechanism module and infusion mechanism module of the infusion device, configured to detect blood glucose continuously. In one embodiment of the present invention, the detection mechanism module 340 is a Continuous Glucose Monitoring (CGM) for detecting real-time BG, monitoring BG changes, and also sending them to the control mechanism module 300.

The control mechanism module 300 is used to control the detection mechanism module 340 and the infusion mechanism module 310. Specifically, the control mechanism module 300 can receive the blood glucose parameter signal sent by the detection mechanism module 340, and is used to control the detection process of the detection mechanism module 340 and record the infusion information and working status of the infusion mechanism module 310. For example, when the blood glucose information detected by the detection mechanism module 340 after the end of life is inaccurate, the control mechanism module 300 may issue a detection stop instruction to the detection mechanism module 340. For another example, when insulin blockage occurs in the infusion mechanism module 310, the control mechanism module 300 can record the blockage status in time and provide feedback to the patient to eliminate potential safety hazards. Therefore, the control mechanism module 300 is connected to the detection mechanism module 340 and the infusion mechanism module 310, respectively. Here, the connection refers to a conventional electrical connection or a wireless connection.

The infusion mechanism module 310 includes the essential mechanical parts used to infuse insulin and controlled by the control mechanism module 300. According to the current insulin infusion dose calculated by the control mechanism module 300, the infusion mechanism module 310 injects the currently insulin dose required into the user's body. At the same time, the real-time infusion status of the infusion mechanism module 310 can also be fed back to the control mechanism module 300.

The embodiment of the present invention does not limit the specific positions and connection or integration relationships of the detection mechanism module 340, the control mechanism module 300 and the infusion mechanism module 310, as long as the aforementioned functional conditions can be satisfied.

As in an embodiment of the present invention, the control mechanism module 300 and the infusion mechanism module 310 are electrically connected or integrated with each other to form a single part while the detection mechanism module 340 is separately provided in another part. At this time, the detection mechanism module 340 and the control mechanism module 300 transmit wireless signals to each other to realize mutual connection. Therefore, the control mechanism module 300 and the infusion mechanism module 310 can be attached on the same position of the user's skin while the detection mechanism module 340 is attached on the other position.

As in another embodiment of the present invention, the control mechanism module 300 and the detection mechanism module 340 are electrically connected or integrated with each other forming a single part while the infusion mechanism module 310 is separately provided in another part. The infusion mechanism module 310 and the control mechanism module 300 transmit wireless signals to each other to realize mutual connection. Therefore, the control mechanism module 300 and the detection mechanism module 340 can be attached on the same position of the user's skin while the infusion mechanism module 310 is attached on the other position.

As in another embodiment of the present invention, the infusion mechanism module 310 and the detection mechanism module 340 are electrically connected or integrated with each other forming a single part while the control mechanism module 300 is separately provided in another part. The infusion mechanism module 310, the detection mechanism module 340 and the control mechanism module 300 transmit wireless signals to each other to realize mutual connection. Therefore, the infusion mechanism module 300 and the detection mechanism module 340 can be attached on the same position of the user's skin while the control mechanism module 300 is attached on the other position or independent of the user's skin, that is, it is not pasted on any part of the user's skin.

As in an embodiment of the present invention, the three are electrically connected or integrated with each other forming a single part. Therefore, the three modules can be attached together on only one position of the user's skin. If the three modules are attached in the only one position, the number of the device on the user skin will be reduced, thereby reducing the interference of more attached devices on user activities. At the same time, it also effectively solves the problem of the poor wireless communication between separating devices, further enhancing the user experience.

As in another embodiment of the present invention, the three are respectively provided in different mechanism modules, thus being attached on different position. At this time, the control mechanism module 300, the detection mechanism module 340 and the infusion mechanism module 310 respectively transmit wireless signals to each other to realize mutual connection.

It should be noted that the control mechanism module 300 of the embodiment of the present invention also has functions such as storage, recording, and access to the database, thus, the control mechanism module 300 can be reused. In this way, not only can the user's physical condition data be stored, but also the production cost and the user's consumption cost can be saved. As described above, when the service life of the detection mechanism module 340 or the infusion mechanism module 310 expires, the control mechanism module 300 can be separated from the detection mechanism module 340, the infusion mechanism module 310, or both the detection mechanism module 340 and the infusion mechanism module 310.

Generally, the service lives of the detection mechanism module 340, the control mechanism module 300 and the infusion mechanism module 310 are different. Therefore, when the three are electrically connected to each other to form a single device, the three can also be separated from each other in pairs. For example, if one module expires firstly, the user can only replace this module and keep the other two modules continuous using.

Here, it should be noted that the control mechanism module 300 of the embodiment of the present invention may also include multiple sub-modules. According to the functions of the sub-modules, different sub-modules can be respectively assembled in different mechanism module, which is not specific limitation herein, as long as the control conditions of the control mechanism module 300 can be satisfied.

As a summary, the present invention discloses a drug infusion device with integrated power supply and the artificial pancreas thereof, the power supply shell is integrated with the lower case and/or the cover plate is integrated with the upper case. The shape and size of the power supply are no longer restricted by the shape and size of the button battery shell, and there is no need for a separate shell. The intergrated power supply occupies a small volume, and more active materials can be accommodated to increase the battery capacity, which can further reduce the volume of the infusion device and the artificial pancreas thereof, while meeting the long-term working requirements of the infusion device and the artificial pancreas thereof, and improve the user experience.

While the invention has been described in detail regarding the specific embodiments of the present invention, it should be understood that it will be appreciated by those skilled in the art that the above embodiments may be modified without departing from the scope and spirit of the invention. The appended claims define the scope of the invention.

1. A drug infusion device with integrated power supply, comprising:
a drug reservoir, used for accommodating a drug to be infused, provided with a piston and a screw;
at least one driving wheel, connected with the screw, driving the screw to push the piston forward by rotation;
a power supply, used to supply power to the drug infusion device, including a power supply shell, a battery cell, electrolyte and a cover plate; and
a case, including an upper case and a lower case, for accommodating the drug reservoir, the drive wheel, and the power supply, wherein the power supply shell is integrated with the lower case and/or the cover plate is integrated with the upper case.

2. The drug infusion device with integrated power supply of claim 1, wherein
an electrolyte isolation layer is arranged on an inside of the power supply shell and the cover plate.

3. The drug infusion device with integrated power supply of claim 2, wherein
the electrolyte isolation layer is a coated TPE or PET layer.

4. The drug infusion device with integrated power supply of claim 3, wherein
a thickness of the electrolyte isolation layer is 300 μm-500 μm.

5. The drug infusion device with integrated power supply of claim 2, wherein
the electrolyte isolation layer is a separated TPE or PET layer.

6. The drug infusion device with integrated power supply of claim 1, wherein
a junction between the power supply shell and the cover plate is coated with an insulating sealing material.

7. The drug infusion device with integrated power supply of claim 6, wherein
the insulating sealing material is hot melt glue or silica gel.

8. The drug infusion device with integrated power supply of claim 1, wherein
the battery cell includes a positive electrode sheet, a negative electrode sheet, a separator, a positive electrode tab and a negative electrode tab, the positive electrode tab is fixedly connected to the positive electrode sheet, the negative electrode tab is fixedly connected to the negative electrode sheet.

9. The drug infusion device with integrated power supply of claim 8,
further comprising a three-dimensional circuit, wherein the positive electrode sheet tab and the negative electrode sheet tab are electrically connected to the three-dimensional circuit, to realize power supply to the drug infusion device.

10. The drug infusion device with integrated power supply of claim 9, wherein
when the cover plate is covering on the power supply shell, a part of the positive electrode tab and the negative electrode tab are reserved out of the power supply shell to be electrically connected to the three-dimensional circuit.

11. The drug infusion device with integrated power supply of claim 9, wherein
two small holes are provided on the power supply shell, and the positive electrode tab and the negative electrode tab are electrically connected to the three-dimensional circuit through the small holes.

12. The drug infusion device with integrated power supply of claim 9,
further comprising a frame, wherein the at least one driving wheel comprises driving wheels, the frame is at least for carrying the driving wheels.

13. The drug infusion device with integrated power supply of claim 12, wherein
the three-dimensional circuit is coated on the frame.

14. The drug infusion device with integrated power supply of claim 12, wherein
the three-dimensional circuit is embedded in the frame.

15. The drug infusion device with integrated power supply of claim 9, wherein
the three-dimensional circuit is coated on the upper case and/or the lower case.

16. The drug infusion device with integrated power supply of claim 9, wherein
the three-dimensional circuit is embedded in the upper case and/or the lower case.

17. The drug infusion device with integrated power supply of claim 1,
further comprising an infusion mechanism module and a control mechanism module, wherein the drug reservoir, the drive wheel, and the power supply are provided on the infusion mechanism module.

18. The drug infusion device with integrated power supply of claim 17, wherein
the infusion mechanism module and the control mechanism module are detachable to each other, and the control mechanism module is reusable.

19. The drug infusion device with integrated power supply of claim 17, wherein
the infusion mechanism module and the control mechanism module are disposed of in one housing, discarded together after a single-use.

20. An artificial pancreas, comprising:
the drug infusion device with integrated power supply of claim 17; and
a detection mechanism module, configured to detect blood glucose continuously, connected or integrated with the control mechanism module and the infusion mechanism module of the drug infusion device.

21. (canceled)

22. (canceled)
